(19) United States
(12) Patent Application Publication
(10) Pub. No.: US 2021/0333214 A1
(43) Pub. Date: Oct. 28, 2021

(54) SYSTEM FOR DETECTION OF A TARGET ANALYTE VIA SELF-TESTING, OBJECT SURFACES, AND THE ENVIRONMENT

(71) Applicant: LogicInk Corporation, San Francisco, CA (US)

(72) Inventors: Carlos Edel OLGUIN ALVAREZ, San Francisco, CA (US); Steven WROBEL, Oakland, CA (US); Hendrik DIETZ, Haar (DE)

(21) Appl. No.: 17/219,527

(22) Filed: Mar. 31, 2021

Related U.S. Application Data
(60) Provisional application No. 63/002,960, filed on Mar. 31, 2020.

(51) Int. Cl. G01N 21/78; G01N 27/414; G01N 21/27

(52) U.S. Cl. CPC G01N 21/78; G01N 2021/773; G01N 21/274; G01N 27/4145

(57) ABSTRACT

Systems and indicators for determining the presence or absence of specific environmental, exposure, or biological conditions are provided. Indicators include a plurality of sensors, each sensor independently having a biological or chemical sensing modality to detect one or more analytes of interest. Analytes of interest include nucleic acids (e.g., DNA, RNA, etc.), proteins, peptides, and other amino acid chains and may come from a subject or the microbiome of a subject. The signals from the plurality of sensors may be processed to provide a readily understandable readout concerning a health condition or predisposition of a subject, such as cancer and exposure to coronavirus. The signals from the plurality of sensors may be colorimetric (e.g. a color change in response to the presence or absence of an analyte), and a plurality of colorimetric signals may be combined to provide a readily understandable colorimetric output. Indicators may be wearable.

### CROSS-REFERENCE TO RELATED APPLICATIONS

This application is related to the following application and claims the benefit of and priority to U.S. Provisional Application No. 63/002,960, filed Mar. 31, 2020, entitled SYSTEM FOR DETECTION OF A TARGET ANALYTE VIA SELF-TESTING, OBJECT SURFACES, AND THE ENVIRONMENT. This application is included in the attached appendix and incorporated by reference in its entirety.

### BACKGROUND

### Field of the Invention

This application generally relates to an indicator comprising sensors that may be wearable or applied to a surface to provide a readily understandable readout indicating a condition of interest.

### Description of Related Art

A person is exposed to a range of environments on a daily basis. The conditions of these environments and the length of exposure to these conditions may impact a person's mental and/or physical state. Several of these conditions may go undetected. Further, their impact on a person exposed to these conditions are not immediately apparent.

For example, ultraviolet wavelengths and particulate contaminates in the air are largely invisible. As another example, the microbiome present on a person's skin may be indicative of the individual's health and is also not immediately apparent.

Additionally, human bodies are continuously exposed to microbial cells and their byproducts which can include toxic metabolites. Circulation of toxic metabolites may contribute to the onset of cancer. In addition, microbes may migrate throughout the human body and become associated with tumor development. Several metagenomics studies have shown that dysbiosis in the commensal microbiota is associated with inflammatory disorders and various cancers. The most recognizable link identified is that between the microbiome and cancer via the immune system. Wen-Ming Wang, Hong-Zhong Jin, Skin microbiome: An actor in the pathogenesis of psoriasis, Chin Med J (Engl). 2018: 131(1), 95-98.

Many associations of gut microflora have been found to be with gastrointestinal cancer. The most prominent association is between (1) Helicobacter pylori and (2) gastric adenocarcinoma and gastric mucosaassociated lymphoid tissue lymphoma. The bacteria Campylobacter jejuni and Salmonella typhi have also been associated with small intestine lymphoma and gall bladder cancer, respectively. In these cases, chronic inflammation at the tumor site induces carcinogenesis.

Gut microflora may play protective roles against cancer as well. Helicobacter pylori has been shown to reduce the risk of esophageal squamous cell carcinoma, and pancreatic cancer. This suggests that a balanced and precise monitoring of microbiome is necessary for a robust immune response.

Thus, it would be beneficial to identify and analyze the microbiome of a subject (or nucleic acids and/or proteins thereof) to be used to predict via correlation the occurrence of carcinogenic conditions, inflammation disorders, and other health conditions.

Additionally, it has been shown that the proportion of the phylum Actinobacteria and the genus Propionibacterium significantly decreased with increasing skin hydration levels on the forehead. Souvik Mukherjee, Rupak Mitra, Arindam Maitra, Satyaranjan Gupta, Srikala Kumaran, Amit Chakrabortty, Partha P. Majumder, Sebum and hydration levels in specific regions of human face significantly predict the nature and diversity of facial skin microbiome, Sci Rep. 2016: 6, 36062. Mukherjee et al. measured sebum and hydration from forehead and cheek regions of healthy female volunteers (n=30) and sequenced metagenomic DNA from skin. 34 phyla were identified—mostly Actinobacteria, Firmicutes, Proteobacteria and Bacteroidetes—and 1000 genera were identified—mostly Propionibacterium, Staphylococcus, Streptococcus, Corynebacterium, and Paracoccus. The analysis showed that cheek sebum levels were the most significant predictors of microbiome composition and diversity followed by forehead hydration levels. These studies showed that the nature and diversity of facial skin microbiome should be determined by site-specific lipid and water levels.

Thus, it would be beneficial to identify and analyze the microbiome of a subject (or nucleic acids and/or proteins thereof) to be used to predict various skin conditions including hydration, serum, and sebum levels.

It has been shown that the skin microbiome greatly impacts a subject's immune functions. In humans, the mechanisms probably include inhibiting the growth of pathogenic microbes, enhancing host innate immunity, and educating adaptive immunity. Elizabeth A. Grice and Julia A. Segre, The skin microbiome, Nature Reviews Microbiology 2011: 9, 244-253. It has been shown that S. epidermidis can inhibit S. aureus biofilm formation. In one study, after inoculation of the upper arm, swabs were taken at multiple time points for Haemophilus ducreyi. Papules either spontaneously resolved or progressed to pustules, with the microbiomes differing between the two groups. Proteobacteria, Bacteroidetes, Micrococcus, Corynebacterium, Paracoccus, and Staphylococcus species were more abundant at pustule-forming sites, whereas resolved sites had a greater abundance of Actinobacteria and Propionibacterium species. These data illustrate a crucial role for commensal bacteria in the host immune defense against pathogens and the importance of using wearable sensor for monitoring them.

Thus, it would be beneficial to identify and analyze the microbiome of a subject (or nucleic acids and/or proteins thereof) to evaluate skin inflammation.

Additionally, recent studies have shown that the presence of human skin microbiome and their composition is directly related to many disorders such as atopic dermatitis, psoriasis, and acne vulgaris. Elizabeth A. Grice, The skin microbiome: potential for novel diagnostic and therapeutic approaches to cutaneous disease, Semin Cutan Med Surg. 2014: 33(2), 98-103. Thus, it would be beneficial to identify and analyze the microbiome of a subject (or nucleic acids and/or proteins thereof) to evaluate skin disorders including acne, psoriasis, and eczema.

The influence of microbiomes on various dermatologic diseases has been investigated by sequencing the 16S rRNA-gene to analyze the correlation of skin bacterial microbiome in several skin disease states, including psoriasis and skin ulcers. Heidi H. Kong, Skin microbiome: genomics-based insights into the diversity and role of skin microbes, Trends Mol Med. 2011, 17(6), 320-328. These studies revealed that toxigenic strains were significantly increased in patients with skin disorders compared to healthy controls. Thus, it would be beneficial to identify and analyze the microbiome of a subject (or nucleic acids and/or proteins thereof) to evaluate the presence of a genetic predisposition towards medical disorders.

Alteration have been observed in the skin microbiome of a subject correlated with aging. Such alterations have been identified by analyzing bacterial 16S rRNA gene sequencing. These analyses revealed that the alpha species was significantly higher in the older than the younger individuals, while the beta diversity in the overall structure significantly differed particularly for the forearm and scalp microbiomes between two age groups. In addition, taxonomic profiling showed a significant reduction in the relative abundance of the majority skin genus Propionibacterium in the cheek, forearm, and forehead microbiomes of the older adults. Nakako Shibagaki, Wataru Suda, Cecile Clavaud, Philippe Bastien, Lena Takayasu, Erica Iioka, Rina Kurokawa, Naoko Yamashita, Yasue Hattori, Chie Shindo, Lionel Breton, Masahira Hattori, Aging-related changes in the diversity of women's skin microbiomes associated with oral bacteria, Sci Rep. 2017: 7, 10567. Thus, it would be beneficial to identify and analyze the microbiome of a subject (or nucleic acids and/or proteins thereof) across the life of the individual and/or with respect to a population to determine age related changes.

Another immediately pressing example of an environmental condition that is not immediately apparent is the presence of viral components, such as those of the corona virus (e.g., COVID-19). Thus, it would be beneficial to identify and analyze the presence of viral components to determine exposure to such components.

Despite the above needs, state-of-the-art sensors are bulky, battery-powered, expensive, and difficult to apply and read. Thus, there is a need for technology capable of readily identifying and analyzing a condition or exposure of interest. Such a sensor would be highly sensitive, specific, low-cost, instrument-free, capable to work at room or body temperature, and/or wearable.

The present technology is directed to indicators comprising a biochemical or chemical set of sensors which are combined to provide a readily understandable readout indicating markers found on a person's body or in the person's environment.

### BRIEF SUMMARY OF INVENTION

One aspect of the invention involves an indicator comprising a substrate; and an interface disposed on the substrate, the interface comprising: a first sensor responsive to a first analyte, the first sensor comprising a first sensing modality, the first sensing modality being a biochemical modality or a chemical modality, and the first sensor being configured to provide a first colorimetric signal upon interaction with the first analyte; a second sensor responsive to a second analyte, the second sensor comprising a second sensing modality, the second sensing modality being a biochemical modality or a chemical modality, and the second sensor being configured to provide a second colorimetric signal upon interaction with the second analyte; and a display configured to display a colorimetric readout.

In one embodiment, the interface is configured to combine the first colorimetric signal and the second colorimetric signal to output the colorimetric readout. The indicator of claim 2, wherein the interface is configured to compound the first colorimetric signal. In one embodiment, the interface is configured to dilute the first colorimetric signal. In one embodiment, the colorimetric readout is substantially identical to the first colorimetric signal. In one embodiment, the colorimetric readout is distinct from the first colorimetric signal, and wherein the colorimetric readout is distinct from the second colorimetric signal. In one embodiment, the colorimetric readout is configured to have an intensity, wherein the intensity is proportional to one or more of the concentration of the first analyte, the concentration of the second analyte, the amount of the first analyte, and the amount of the second analyte.

In some embodiments, the substrate is a polymeric substrate. In one embodiment, the colorimetric readout is configured as a single colorimetric readout.

In one embodiment, the first sensing modality is a cell-free modality, a whole-cell modality, or a nanoparticle modality. In some embodiments, the first analyte is single-stranded DNA.

In one embodiment, the interface comprises a logical gate, the logic gate is configured to output the colorimetric readout, wherein the logic gate is responsive to a predetermined logical condition. In one embodiment, the predetermined logical condition is at least one of the first colorimetric signal and the second colorimetric signal being a Boolean true signal. In some embodiments, the predetermined logical condition is at least one of the first colorimetric signal and the second colorimetric signal being a Boolean false signal. In one embodiment, the predetermined logical condition is the first colorimetric signal being a Boolean true signal and the second colorimetric signal being a Boolean false signal. In some embodiments, the indicator further comprises a second logic gate, wherein the second logic gate is responsive to a second predetermined logical condition, and wherein the first logic gate and the second logic gate are configured to output the colorimetric readout.

In one embodiment, the first analyte is an antigen, wherein the first sensing modality comprises one or more antibodies, and wherein the antigen binds to the one or more antibodies. In one embodiment, the first analyte is an antibody, wherein the first sensing modality comprises one or more antigens, and wherein the antigen binds to the one or more antibodies.

In one embodiment, the first analyte is a nucleic acid, wherein the first sensing modality comprises one or more nucleic acids, wherein the first analyte interacts with the one or more nucleic acids of the first sensing modality, and wherein the first analyte interacts with the one or more nucleic acids of the first sensing modality based on one or more of intercalating agents, enzymes, beacons, or salts. In one embodiment, the first analyte interacts with the one or more nucleic acids of the sensing modality based on enzymes, and wherein at least one of the one or more nucleic acids of the first sensing modality has a G-hairpin conformation. In one embodiment, wherein at least one of the first analyte and the second analyte is amplified before the at least one of the first analyte and the second analyte is sensed by at least one of the first sensor and second sensor.

In one embodiment, the at least one of the first analyte and the second analyte is a nucleic acid. In one embodiment, the first sensing modality comprises one or more bioreceptors. In one embodiment, the first sensing modality comprises one or more nucleic acids. In one embodiment, at least one of the one or more nucleic acids is obtained from an engineered organism. In one embodiment, the first sensing modality further comprises nanomaterials, wherein the nanomaterials constitute a host matrix, and wherein the one or more bioreceptors are disposed on the host matrix. In one embodiment, the nanomaterials are carbon nanomaterials.

In one embodiment, the first sensor is in fluid communication with the second sensor. In one embodiment, the first sensor is not separated from the second sensor. In one embodiment, the indicator is configured to be wearable on the skin of a subject. In one embodiment, the indicator is configured to be disposed on a surface. In one embodiment, the indicator further comprises an adhesive layer.

In one embodiment, the indicator further comprises a membrane layer. In one embodiment, the indicator further comprises an adhesive layer. In one embodiment, the membrane layer is porous. In one embodiment, the membrane layer comprises a nanomaterial.

In one embodiment, the first analyte is derived from a microbiome of a subject. In one embodiment, the presence or absence of the first analyte correlates with one or more of a skin condition, skin hydration, serum levels, skin inflammation, a skin disorder, acne, psoriasis, eczema, dermatitis, a predetermined genetic predisposition, a genetic predisposition of developing psoriasis, a genetic predisposition of developing a skin ulcer, an age-related condition or change, the presence of a virus, a condition associated with cancer, gastric adenocarcinoma, gastric mucosa-associated lymphoid tissue lymphoma, intestine lymphoma, gall bladder cancer, esophageal squamous cell carcinoma, or pancreatic cancer. In one embodiment, the first analyte is derived from a COVID-19 virion, a Helicobacter pylori cell, a Campylobacter jejuni cell, a Salmonella typhi cell, an organism belonging to the phylum Actinobacteria, an organism belonging to the phylum Firmicutes, an organism belonging to the phylum Proteobacteria, an organism belonging to the phylum Bacteroidetes, an organism belonging to the genus Propionibacterium, an organism belonging to the genus Staphylococcus, an organism belonging to the genus Streptococcus, an organism belonging to the genus Corynebacterium, an organism belonging to the genus Paracoccus, an S. epidermidis cell, an S. aureus cell, an Haemophilus ducreyi cell, or an organism belonging to the phylum Micrococcus.

In one embodiment, the first analyte and the second analyte are different analytes. In one embodiment, the first analyte and the second analyte are the same analyte.

One aspect of the invention involves a method for determining exposure to at least one analyte, the method comprising: providing an indicator comprising a substrate; an interface disposed on the substrate, the interface comprising: a first sensor responsive to a first analyte, the first sensor comprising a first sensing modality, the first sensing modality being a biochemical modality or a chemical modality, and the first sensor being configured to provide a first colorimetric signal upon interaction with the first analyte; a second sensor responsive to a second analyte, the second sensor comprising a second sensing modality, the second sensing modality being a biochemical modality or a chemical modality, and the second sensor being configured to provide a second colorimetric signal upon interaction with the second analyte; and a display configured to display a colorimetric readout; determining exposure to at least one of the first analyte and the second analyte; and displaying the colorimetric readout on the display.

In one embodiment, the interface is configured to combine the first colorimetric signal and the second colorimetric signal to output the colorimetric readout.

In one embodiment, the interface is configured to combine the first colorimetric signal and the second colorimetric signal to output the colorimetric readout. In one embodiment, the interface is configured to compound the first colorimetric signal. In one embodiment, the interface is configured to dilute the first colorimetric signal. In one embodiment, the colorimetric readout is substantially identical to the first colorimetric signal. In one embodiment, the colorimetric readout is distinct from the first colorimetric signal, and wherein the colorimetric readout is distinct from the second colorimetric signal. In one embodiment, the colorimetric readout is configured to have an intensity, wherein the intensity is proportional to one or more of the concentration of the first analyte, the concentration of the second analyte, the amount of the first analyte, and the amount of the second analyte.

In one embodiment, the substrate is a polymeric substrate. In one embodiment, the step of determining exposure to at least one of the first analyte and the second analyte comprises accessing the colorimetric readout.

In one embodiment, the step of determining exposure to at least one of the first analyte and the second analyte comprises determining the approximate real-time presence or absence of at least one of the first analyte and the second analyte. In one embodiment, the display of the colorimetric readout is reversible and further comprises a step of eliminating the colorimetric readout from the display.

In one embodiment, the step of determining exposure to at least one of the first analyte and the second analyte comprises determining the cumulative exposure to at least one of the first analyte and the second analyte. In one embodiment, the colorimetric readout is irreversible.

In one embodiment, the colorimetric readout is a colorimetric readout.

In one embodiment, the first sensing modality is a cell-free modality, a whole-cell modality, or a nanoparticle modality. In one embodiment, the first analyte is single-stranded DNA.

In one embodiment, the interface comprises a logical gate, the logic gate configured to output the colorimetric readout, wherein the logic gate is responsive to a predetermined logical condition. In one embodiment, the predetermined logical condition is at least one of the first colorimetric signal and the second colorimetric signal being a Boolean true signal. In one embodiment, the predetermined logical condition is at least one of the first colorimetric signal and the second colorimetric signal being a Boolean false signal. In one embodiment, the predetermined logical condition is the first colorimetric signal being a Boolean true signal and the second colorimetric signal being a Boolean false signal. In one embodiment, the indicator further comprises a second logic gate, wherein the second logic gate is responsive to a second predetermined logical condition, and wherein the first logic gate and the second logic gate are configured to output the colorimetric readout.

In one embodiment, the first analyte is an antigen, wherein the first sensing modality comprises one or more antibodies, and wherein the antigen binds to the one or more antibodies. In one embodiment, the first analyte is an antibody, wherein the first sensing modality comprises one or more antigens, and wherein the antigen binds to the one or more antibodies.

In one embodiment, the first analyte is a nucleic acid, wherein the first sensing modality comprises one or more nucleic acids, wherein the first analyte interacts with the one or more nucleic acids of the first sensing modality, and wherein the first analyte interacts with the one or more nucleic acids of the first sensing modality based on one or more of intercalating agents, enzymes, beacons, or salts. In one embodiment, the first analyte interacts with the one or more nucleic acids of the sensing modality based on enzymes, and wherein at least one of the one or more nucleic acids of the first sensing modality has a G-hairpin conformation.

In one embodiment, the method further comprises a step of amplifying at least one of the first analyte and the second analyte. In one embodiment, the at least one of the first analyte and the second analyte is a nucleic acid. In one embodiment, the first sensing modality comprises one or more bioreceptors. In one embodiment, the first sensing modality comprises one or more nucleic acids. In one embodiment, at least one of the one or more nucleic acids is obtained from an engineered organism. In one embodiment, the first sensing modality further comprises nanomaterials, wherein the nanomaterials constitute a host matrix, and wherein the one or more bioreceptors are disposed on the host matrix. In one embodiment, the nanomaterials are carbon nanomaterials.

In one embodiment, the first sensor is in fluid communication with the second sensor. In one embodiment, the first sensor is not separated from the second sensor. In one embodiment, the indicator is configured to be wearable on the skin of a subject. In one embodiment, the indicator is configured to be disposed on a surface. In one embodiment, the indicator further comprises an adhesive layer.

In one embodiment, the indicator further comprises a membrane layer. In one embodiment, the indicator further comprises an adhesive layer. In one embodiment, the membrane layer is porous. In one embodiment, the membrane layer comprises a nanomaterial.

In one embodiment, the first analyte is derived from a microbiome of a subject. In one embodiment, the method further comprises the step of determining a condition of the subject, wherein the condition is one or more of a skin condition, the skin hydration of the subject, the serum levels of the subject, skin inflammation, a skin disorder, acne, psoriasis, eczema, dermatitis, a predetermined genetic predisposition, a genetic predisposition of developing psoriasis, a genetic predisposition of developing a skin ulcer, an age-related condition or change, viral exposure, a condition associated with cancer, gastric adenocarcinoma, gastric mucosa-associated lymphoid tissue lymphoma, intestine lymphoma, gall bladder cancer, esophageal squamous cell carcinoma, or pancreatic cancer. In one embodiment, the method further comprises the step of determining the presence of COVID-19, Helicobacter pylori, Campylobacter jejuni, Salmonella typhi, an organism belonging to the phylum Actinobacteria, an organism belonging to the phylum Firmicutes, an organism belonging to the phylum Proteobacteria, an organism belonging to the phylum Bacteroidetes, an organism belonging to the genus Propionibacterium, an organism belonging to the genus Staphylococcus, an organism belonging to the genus Streptococcus, an organism belonging to the genus Corynebacterium, an organism belonging to the genus Paracoccus, an S. epidermidis cell, an S. aureus cell, an Haemophilus ducreyi cell, or an organism belonging to the phylum Micrococcus.

In one embodiment, the first analyte and the second analyte are different analytes. In one embodiment, the first analyte and the second analyte are the same analyte.

One aspect of the invention involves a system for determining exposure to at least one analyte, the system comprising: an indicator comprising a substrate; and an interface disposed on the substrate, the interface comprising: a first sensor responsive to a first analyte, the first sensor comprising a first sensing modality, the first sensing modality being a biochemical modality or a chemical modality, and the first sensor being configured to provide a first signal upon interaction with the first analyte; a second sensor responsive to a second analyte, the second sensor comprising a second sensing modality, the second sensing modality being a biochemical modality or a chemical modality, the second sensor being configured to provide a second signal upon interaction with the second analyte; and a reactive solution; wherein the reactive solution is configured to interact with one or more of the first signal and the second signal to produce a colorimetric readout.

In one embodiment, the colorimetric readout has an intensity, wherein the intensity is proportional to one or more of the concentration of the first analyte, the concentration of the second analyte, the amount of the first analyte, and the amount of the second analyte. In one embodiment, the substrate is a polymeric substrate. In one embodiment, the colorimetric signal is displayed on the display. In one embodiment, the colorimetric signal is a change in the color of the reactive solution.

In one embodiment, the first sensing modality is a cell-free modality, a whole-cell modality, or a nanoparticle modality. In one embodiment, the first analyte is single-stranded DNA.

In one embodiment, the indicator comprises a logical gate, the logic gate is configured to output the processed signal, wherein the logic gate is responsive to a predetermined logical condition. In one embodiment, the predetermined logical condition is at least one of the first signal and the second signal being a Boolean true signal. In one embodiment, the predetermined logical condition is at least one of the first signal and the second signal being a Boolean false signal. In one embodiment, the predetermined logical condition is the first signal being a Boolean true signal and the second signal being a Boolean false signal. In one embodiment, the indicator further comprises a second logic gate, wherein the second logic gate is responsive to a second predetermined logical condition, and wherein the first logic gate and the second logic gate are configured to output the processed signal.

In one embodiment, the first analyte is an antigen, wherein the first sensing modality comprises one or more antibodies, and wherein the antigen binds to the one or more antibodies. In one embodiment, the first analyte is an antibody, wherein the first sensing modality comprises one or more antigens, and wherein the antigen binds to the one or more antibodies.

In one embodiment, the first analyte is a nucleic acid, wherein the first sensing modality comprises one or more nucleic acids, wherein the first analyte interacts with the one or more nucleic acids of the first sensing modality, and wherein the first analyte interacts with the one or more nucleic acids of the first sensing modality based on one or more of intercalating agents, enzymes, beacons, or salts. In one embodiment, the first analyte interacts with the one or more nucleic acids of the sensing modality based on enzymes, and wherein at least one of the one or more nucleic acids of the first sensing modality has a G-hairpin conformation.

In one embodiment, the system further comprises an amplifier to amplify at least one of the first analyte and the second analyte. In one embodiment, the at least one of the first analyte and the second analyte is a nucleic acid. In one embodiment, the first sensing modality comprises one or more bioreceptors. In one embodiment, the first sensing modality comprises one or more nucleic acids. In one embodiment, at least one of the one or more nucleic acids is obtained from an engineered organism. In one embodiment, the first sensing modality further comprises nanomaterials, wherein the nanomaterials constitute a host matrix, and wherein the one or more bioreceptors are disposed on the host matrix. In one embodiment, the nanomaterials are carbon nanomaterials.

In one embodiment, the first sensor is in fluid communication with the second sensor. In one embodiment, the first sensor is not separated from the second sensor. In one embodiment, the indicator is configured to be wearable on the skin of a subject. In one embodiment, the indicator is configured to be disposed on a surface. In one embodiment, the indicator further comprises an adhesive layer.

In one embodiment, the indicator further comprises a membrane layer. In one embodiment, the indicator further comprises an adhesive layer. In one embodiment, the membrane layer is porous. In one embodiment, the membrane layer comprises a nanomaterial.

In one embodiment, the first analyte is derived from a microbiome of a subject. In one embodiment, the presence or absence of the first analyte correlates with one or more of a skin condition, skin hydration, serum levels, skin inflammation, a skin disorder, acne, psoriasis, eczema, dermatitis, a predetermined genetic predisposition, a genetic predisposition of developing psoriasis, a genetic predisposition of developing a skin ulcer, an age-related condition or change, the presence of a virus, a condition associated with cancer, gastric adenocarcinoma, gastric mucosa-associated lymphoid tissue lymphoma, intestine lymphoma, gall bladder cancer, esophageal squamous cell carcinoma, or pancreatic cancer. In one embodiment, the first analyte is derived from a COVID-19 virion, a Helicobacter pylori cell, a Campylobacter jejuni cell, a Salmonella typhi cell, an organism belonging to the phylum Actinobacteria, an organism belonging to the phylum Firmicutes, an organism belonging to the phylum Proteobacteria, an organism belonging to the phylum Bacteroidetes, an organism belonging to the genus Propionibacterium, an organism belonging to the genus Staphylococcus, an organism belonging to the genus Streptococcus, an organism belonging to the genus Corynebacterium, an organism belonging to the genus Paracoccus, an S. epidermidis cell, an S. aureus cell, an Haemophilus ducreyi cell, or an organism belonging to the phylum Micrococcus.

In one embodiment, the first analyte and the second analyte are different analytes. In one embodiment, the first analyte and the second analyte are the same analyte.

### BRIEF DESCRIPTION OF THE DRAWINGS

FIG. 1 depicts an exemplary logic gate that may be employed in certain embodiments of the instant technology.

FIG. 2 depicts exemplary visual indicators of output variants that provide a qualitative signal proportional to the strength of the condition of interest or analyte being sensed.

FIGS. 3A-3C depict exemplary sensors that are prevented from coming into direct contact with a test sample or environment by application of a substrate layer. FIG. 3A depicts a folded indicator. FIGS. 3B and 3C depict indicators without folding.

FIGS. 4A-4E depict exemplary embodiments of the technology. The indicator may be removably applied to a subject in the form of a “tattoo.” FIGS. 4A and 4B depict a removable tattoo that, upon removal, may be placed in a reactive solution. FIG. 4A depicts that in the presence of a signal from the tattoo, the solution may change color. FIG. 4B depicts that in the presence of a signal from the tattoo, the tattoo may change color in the solution. FIG. 4C depicts a removable tattoo that presents a directly observable color change due to specific interactions between sensors and analyte(s) of interest while it is applied to the subject. FIG. 4D depicts an indicator upon which a solution comes into contact, and, in the presence of a signal form the tattoo, the tattoo may change color. FIG. 4E depicts a removable tattoo that may change color upon contact with a biological fluid, and, in the presence of a signal, the tattoo may change color. In all of these examples, the signal may be the presence of one or more analytes or a resulting signal resulting from one or more sensors responsive to one or more analytes.

FIG. 5 depicts an exemplary embodiment of the technology wherein the indicator is placed on a high-contact surface.

FIGS. 6A and 6B depict embodiments of the technology wherein at least one of the one or more analytes of interest are nucleic acids. These nucleic acids can be obtained from dead skin cells, the skin microbiome. FIG. 6A depicts the collection of nucleic acids from various sources. FIG. 6B depicts a simplified readout.

FIG. 7 depicts an embodiment of the technology where an object (e.g., a tissue paper) comprising the indicator technology.

FIG. 8 depicts various exemplary embodiments of the technology.

### DETAILED DESCRIPTION

The present technology is directed to indicators comprising a biochemical or chemical set of sensors. These indicators are combined to provide a readily understandable readout indicating specific environmental or exposure conditions. Such conditions may include health conditions or exposure to virulent agents or carcinogens.

The instant indicator technology may include a substrate layer that may be or include an adhesive, an interface that may be disposed on the substrate layer, and a sensor configuration comprising at least 1 or a plurality of sensors. The indicator may further comprise a porous membrane that allows for one or more analyte of interest to pass through. In some embodiments, the technology further comprises a display. The reaction of one or more sensor with one or more analyte may produce a signal to be displayed on the display of the indicator. The displayed signal may include a change in a visual appearance, such as a change in color.

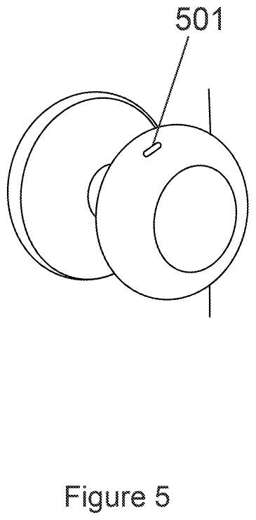

The indicator may be temporarily adhered to a variety of different surfaces. The indicator may be permanently adhered to a surface. The indicator may include one or more adhesive layers to enable adhering the indicator to a surface. The different surfaces including skin, clothing, packaging, surfaces (e.g., doors, doorknobs, walls, tools), or other objects. For example, an indicator 501 may be directly applied to a doorknob as in FIG. 5. The indicator may be place in environments including an outdoor environment and an indoor environment. The indicator may be in the form of a tattoo that can be removed or a test strip.

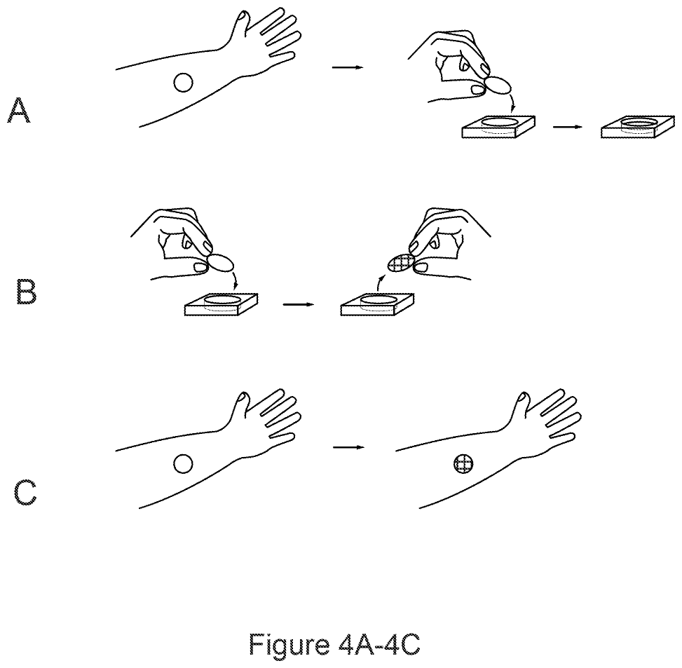

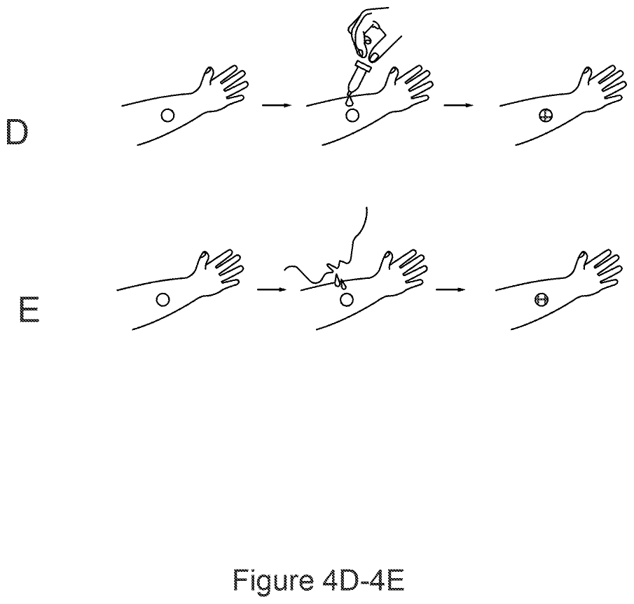

In certain embodiments, the indicator may be part of system further comprising at least one or more appropriate reactive solutions. FIGS. 4A-4E depict exemplary systems wherein the indicator may be removably applied to a subject in the form of a “tattoo.” Reaction with the removable tattoo with an appropriate reactive solution as depicted in FIGS. 4A and 4B may be used to produce a detectable signal, such as a color change. In certain embodiments, as depicted in FIG. 4C, the tattoo prevents a detectable signal without requiring a separate reactive solution.

The substrate of the indicator may be a film, or a polymeric film. The substrate may have a first side and a second side with the interface disposed on the first side or the second side of the substrate.

The interface may provide a display disposed on the first side or on the second side of the substrate. In some embodiments the sensor configuration includes at least one sensor that is directly printed on the surface of the interface. Printing may be performed using any appropriate means, including flexographic printing and inkjet printing. In certain embodiments, the interface is flexible and comes into contract with the substrate by folding the flexible interface on to the substrate.

The sensor configuration may comprise at least 1, 2, 3, 4, 5, 6, 7, 8, 9, 10, or any plurality of sensors. The sensor configuration may comprise at least a first sensor and a second sensor. Each sensor in the sensor configuration may independently have a sensing modality. Each sensing modality may be a biochemical sensor, a chemical sensor, or some combination thereof (e.g., cell-free enzymes). Each sensing modality may be responsive to one or more analytes. Upon contact between a sensing modality and an analyte of interest, the sensing modality and/or the analyte may undergo a reversible or irreversible interaction. A plurality of sensors in the sensing configuration may be in fluid communication with one another. A plurality of sensors in the sensing configuration might not be separated from one another.

In certain embodiments, at least one of the sensors of the sensor configuration comes in direct contact with a sample or the environment. In certain embodiments, a membrane layer separates a sample or the environment from at least one of the sensors of the sensor configuration. The membrane layer may be a nanoporous membrane. The membrane layer may transport various components including reagents, samples, extracted analytes of interest (e.g., nucleic acids such as DNA). The transport provided by the membrane layer may be due to capillary forces. In certain embodiments, the membrane layer provides one or more chamber for isothermal, instrument-free, homogenous analysis of samples at ambient or skin temperature.

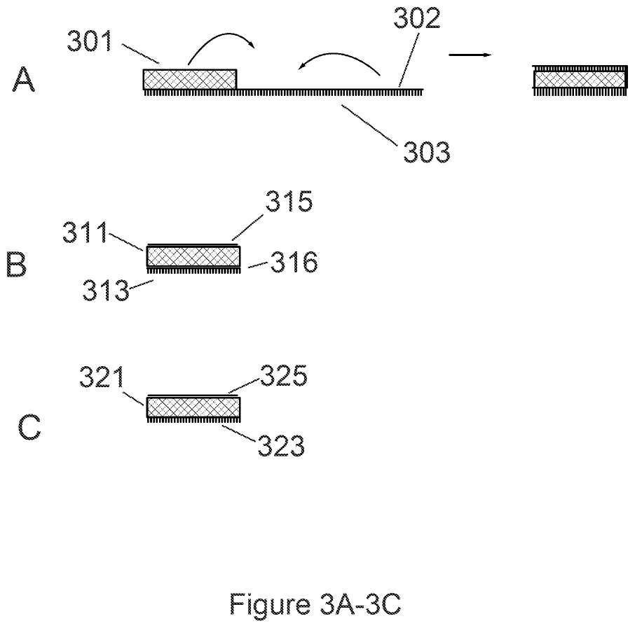

Any appropriate configuration may be used to provide the sensors of the instant technology. FIG. 3A depicts an exemplary sensor that does not come into direct contact with a test sample or environment. The sensor of FIG. 3A comprises a substrate 302 and an adhesive 303 folded onto a sensor 301. The resulting configuration has the adhesive facing inwards contacting the sensor. In another environment, the adhesive 303 comes into contact with a sample (e.g., a subject's skin or clothing). A portion of the sample (e.g., dead skin cells, a portion of the subject's microbiome, etc.) come into contact with the adhesive and adhere to the adhesive. Upon folding of the adhesive 303 onto the sensor 301 the adhered portion of the sample is transferred to the sensor. In another exemplary embodiment depicted by FIG. 3B, a sensor 311 is in contact with an optional superstrate 315 on one face. On another face the sensor is in contact with a substrate layer 316 in contact with an adhesive 313. The substrate may be nanoporous to permit sample to reach the sensor. FIG. 3C depicts a sensor 321 in contact with an adhesive 323 on one face. On another face the sensor is in contact with an optional superstate 325. A sensor may be adhered directly to the sample (e.g., a subject's skin or clothing) by the adhesive. The adhesive may be porous or nanoporous to permit sample to reach the sensor.

Each sensing modality may independently include bioreceptors, biomolecules, or living cells, with high affinities towards one or more analyte of interest with high specificities. In certain embodiments, the bioreceptors are based on a nucleic acid (e.g. single-stranded DNA or RNA). The nucleic acid bioreceptors may target a complementary or partially complementary nucleic acid of interest (e.g., a single-stranded DNA, a double-stranded DNA, or an RNA). The complementary analyte nucleic acids may hybridize to (i.e., form base pairs with) nucleic acid bioreceptors of the sensors to form double stranded. For instance, in the case of a single-stranded DNA bioreceptor nucleic acid and a single-stranded DNA analyte, the strands may hybridize to form double-stranded DNA. Similarly, in a single-stranded DNA bioreceptor nucleic acid and a single-stranded RNA analyte may hybridize to form a double-stranded DNA-RNA construct. It is known in the art how to convert double-stranded nucleic acids into single-stranded nucleic acids for capture or release. In this way, the indicator can sense the presence of one or more targeted nucleic acid.

In certain embodiments, each sensing modality may independently include the integration of nanomaterials as a host matrix. Use of a nanomaterial host matrix may provide an increased capacity or concentration of bioreceptors in a sensor. In this way, a higher density of bioreceptor may be incorporated into a sensor to relatively increase the sensor's sensitivity toward an analyte of interest. In certain embodiments, the nanomaterial is a carbon nanomaterial. Examples of carbon nanomaterials that may be host matrices in the sensors are graphene and carbon nanotubes. Graphene and carbon nanotubes provide a larger surface area to which bioreceptors may be attached resulting in a higher density of bioreceptors.

Each sensing modality may be a cell-free modality, a whole-cell modality, or a nanoparticle modality. In certain embodiments, the sensing modality may be a reaction mixture that produces and processes the signal.

In certain embodiments, the whole-cell modality may provide a signal based on the interaction of receptors on the surface of a whole cell with one or more analytes of interest (e.g., one or more proteins). The interaction of the whole cells and the one or more analytes may be based on strong and irreversible antibody-antigen bindings. In certain embodiments, a complete or partial antibody may be presented on the surface of a whole cell. In certain embodiments, a complete or partial antigen may be presented on the surface of a whole cell. The antibody-antigen interaction may be selective and sensitive. For example, some antibody-antigen interactions are known to bind with high affinity (e.g., Ka of 106).

Nanoparticle modalities include metallic nanoparticles. The metallic nanoparticles may include gold nanoparticles (AuNPs) and/or silver nanoparticles (AgNPs). Certain nanoparticle modalities may be responsive to a nucleic acid (e.g., DNA, single-stranded DNA, double-stranded DNA, RNA, etc.). In certain embodiments, the sensing metallic nanoparticles may sense single-stranded nucleic acids by way of its inhibitory effect on the nanoparticle aggregation. In certain embodiments, the hybridization of single-stranded nucleic acids with other nucleic acids may result in the removal of this inhibitory effect providing an output signal from the sensor. The size of the metallic nanoparticles may be adjusted to be more or less sensitive to concentrations of nucleic acids of interest. For instance, larger nanoparticles aggregate faster even in the presence of low concentrations of analyte, and so the use of larger nanoparticles would result in a sensor more sensitive to lower concentrations of nucleic acids of interest.

In certain embodiments, the aggregation of the metallic nanoparticles (e.g., AuNPs) may be determined by their optical properties. The optical properties may be determined by surface plasmon resonance or another appropriate method to detect changes in nanoparticle aggregation status. The peak absorbance of a surface plasmon resonance may provide information concerning the distance between particles. Once aggregation occurs, the surface plasmon resonance of particles may become coupled and shift the absorbance spectrum. This shift may be large enough to produce a visible color change, which makes the techniques favorable for diagnostics. In the presence of salt, AuNPs may aggregate and change color from red to blue unless they can be stabilized by nucleic acids. Nanoparticle stabilization may be interrupted by formation of double-stranded nucleic acids which may decrease the distance between metallic nanoparticles.

Certain sensing modalities may comprise antigens that are responsive to antibodies. Certain sensing modalities may comprise antibodies that are responsive to antigens. Certain sensing modalities may comprise one or more nucleic acids (e.g., DNA, single-stranded DNA, double-stranded DNA, RNA, etc.). In certain embodiments, sensing modalities comprising one or more nucleic acids may be responsive to a second nucleic acid. Responsiveness to the second nucleic acid may be based on one or more components including one or more intercalating agents, one or more soluble salt, one or more beacons, one or more reversible switching beacons, one or more molecular beacons, one or more enzymes, and one or more hairpins in nucleic acids. For example, the hybridization of a single-stranded nucleic acid of interest to a single-stranded nucleic acid of a sensing modality may be altered by the presence of one or more intercalating agents that weaken the nucleic acid-nucleic acid interaction. By increasing or decreasing the amount or concentration of intercalating agents, the sensitivity of the sensing modalities may be adjusted in certain embodiments. Likewise, a soluble salt may assist in dissociating bonds between base pairs of hybridized nucleic acids (e.g., double-stranded DNAs). By introducing or adjusting soluble salts, the sensitivity of the sensing modalities may be adjusted or maid aid in releasing hybridized target nucleic acids from a sensing modality to recover the nucleic acid of interest. Generally, these salts can contribute to dissociation of bindings between the bioreceptors and the targeted analyte.

In certain embodiments detection of nucleic acids can be performed using one or more reversible switching beacons or enzymes. For example, detection may be performed by a G-quadruplex DNA strand that may be present on a beacon.

A molecular beacon may function as a sensing modality in certain embodiments of the technology. When a donor molecule and an acceptor molecule are in close proximity, the acceptor molecule produces a readable signal. Such embodiments can be enabled using fluorescence resonance energy transfer (FRET) or bioluminescence resonance energy transfer (BRET), which does not require an external light source to excite the fluorophore. This ability to function in the absence of an external light source permits a sensor platform to function as an instrument-free platform or as a platform requiring fewer instruments. This provides the benefits of an increased ease of use, increased ease of transportation and installation, and decreased complexity. In other embodiments, the signal may be created by distancing a quencher molecule from a fluorophore. In certain embodiments, the excitation of a fluorophore may produce an emission in the visible spectrum. In certain embodiments, other modalities or methods may be used to produce parallel signals. For example, a sensor configuration may comprise a sensor comprising a FRET-based sensing modality and a sensor comprising an ELISA-like assay for viral protein detection. In this manner, the indicator may increase capacity through diversification and/or confidence through redundancy.

In certain embodiments, the one or more analytes may be amplified before detection by the indicator. In certain embodiments, the amount of amplification will correlate with the relative amount or concentration of the original analyte. In some embodiments, the amplification will result in the signal resulting from the amplified analyte to dominate the colorimetric readout or processed signal produced.

The amount or concentration may vary across a spatial area to provide a gradient that is detectable by a plurality of spatially separated sensors of the sensor configuration. By retaining the spatial information of the plurality of sensors of the sensor configuration, a determination of the gradient or variance of the one or more analytes can be detected by the indicator.

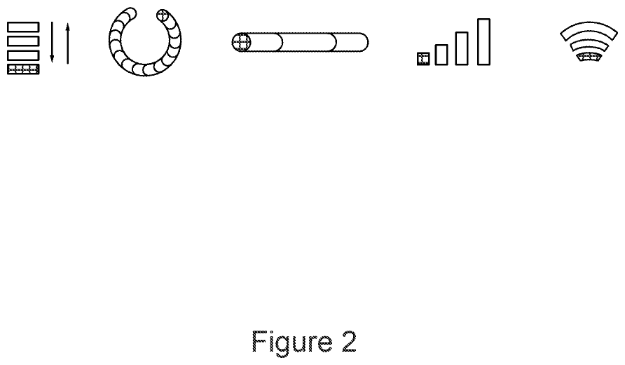

In some embodiments, the amount or concentration of one or more analytes in a sample may be represented in a qualitative fashion. For example, FIG. 2 depicts exemplary visual indicators of output variants that provide a qualitative representation proportional to the strength of the condition of interest or analyte being sensed. For example, each bar can be calibrated to display a signal when at a specified range of signals from the sensors relating to the amount or concentration of one or more analytes. Therefore, the bars will respond differently to a given analyte concentration. For example, in a three-bar embodiment, one bar may display a signal when the analyte concentration or amount is determined to be low or undetectable, two bars may display a signal when the analyte concentration or amount is determined to be moderate, and three bars may display a signal when the analyte concentration or amount is determined to be high. In some embodiments, no bars will provide a signal to indicate the analyte concentration is low, very low, undetectably low, or absent. Thereby the technology may provide the user a scale on which the user can read off analyte concentration.

Amplification of various analytes are known in the art. For example, in an embodiment employing nucleic acid bioreceptors that may target a complementary or partially complementary nucleic acid of interest, the signal from hybridization may be amplified to provide a stronger signal. For example, where the hybridization of the target nucleic acid of interest to a nucleic acid bioreceptor dissociates a quencher moiety from a fluorophore moiety, the binding of one target nucleic acid to one nucleic acid bioreceptor may dissociate multiple fluorophores thereby providing an amplified signal. BS Alladin-Mustan, C J Mitran, J M Gibbs-Davis, Achieving room temperature DNA amplification by dialing in destabilization. Chem Commun (Camb). 2015 Jun. 4; 51(44):9101-4. doi: 10.1039/c5cc01548k. PMID: 25920515. Further examples of amplification and detection are known in the art. Gerasimova, Yulia V., and Dmitry M. Kolpashchikov. “Enzyme-assisted target recycling (EATR) for nucleic acid detection.” Chemical Society Reviews 43.17 (2014): 6405-6438. In other embodiments, the amplification may be the loss fluorescent signals from multiple fluorophores upon their coming into proximity with one or more quencher moieties. In an embodiment, the amplification may be performed using nanomaterials or nanoparticles (e.g., AuNPs, AgNPs, etc.). In certain embodiments, amplification may be isothermal. The amplification may be conducted at ambient (e.g., 22° C.) or near-ambient temperature. The amplification may be performed using an enzyme that operates at skin or near-skin temperatures (e.g., 30° C.). In a certain embodiment, the enzyme may be Phi29.

The indicator is capable of being calibrated to any useful sensitivity of one or more sensor. Calibration of the one or more sensor sensitivities can be done in many ways known in the art including finetuning the stoichiometry of the reagents. In certain embodiments, the indicator is capable of achieving a sensitivity of about 1, 0.5, 0.4, 0.3, 0.25, 0.2, or 0.1 nM of the analyte of interest. In certain embodiments, the sensitivity is about 0.3 nM of the analyte of interest. In certain embodiments, the indicator may have a specificity for a nucleic acid analyte permitting the indicator to distinguish nucleic acids having about one nucleotide substitution per 100, 75, 50, 25, or 10 nucleotides of a nucleic acid. In certain embodiments, the indicator may have a specificity for a nucleic acid analyte permitting the indicator to distinguish nucleic acids having about one nucleotide substitution per 25 nucleotides. In certain embodiments, the indicator may have a specificity for a nucleic acid analyte permitting the indicator to distinguish a 25-nucleotide-long analyte having a single nucleotide substitution.

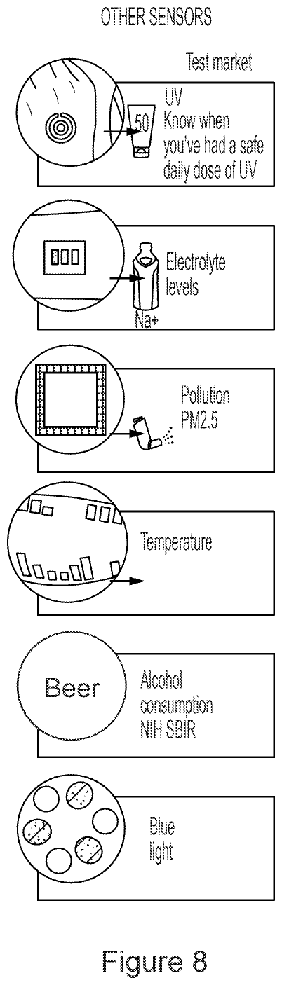

FIG. 8 depicts various other sensors that may be incorporated into the instant indicator technology. One or more sensors of the indicator may be receptive to various environmental conditions such as cumulative UV exposure, electrolyte levels, particulates, temperature, alcohol consumption, and blue light exposure.

Each sensor may be independently responsive to one or more analytes. A sensor responsive to an analyte may provide a signal upon interaction with the analyte. In preferred embodiments, the signals are colorimetric signals. In some embodiment, the sensor may be a negative sensor that provides a signal when an analyte to which it is receptive is not present. In some embodiments, the sensor may provide one colorimetric signal (e.g., red) in the absence of an analyte to which it is receptive and a different colorimetric signal (e.g., blue) in the presence of the analyte. In embodiments where the sensor provides a colorimetric signal in the presence of an analyte and second colorimetric signal in the absence of the analyte, the colorimetric signals may differ in intensity, saturation, vibrancy, opacity, transparency, degree, and/or any other color characteristic, but generally described by the same generic color term (e.g., red and light red/pink; bright blue and dark blue; neon yellow and gold; black and grey; an opaque purple and a transparent/semi-transparent purple).

The signals or absence of signals from a plurality of sensors may be processed with each other. The signals or absence of signals from a plurality of sensors may undergo processing together. The process may include aggregating one or more signals and/or one or more absences of signal, combining one or more signals and/or one or more absences of signal, and/or subjecting one or more signals and/or one or more absences of signal to a function. For example, the enzyme described above may potentially respond to a hybridization event in the loop region but also to a nuclease-driven loop cutting event, which in both cases can lead to restoring the activity of the G quadruplex enzyme, therefore aggregating two different inputs into one output. In some instances, the combination of two colorimetric signals will compound or strengthen at least one of the signals (e.g., light red signals combine to produce a vibrant red signal), will weaken at least one of the signals (e.g., opaque red and transparent red combine to produce semi-transparent red; red and another signal combine to produce a light red/pink), or will produce a colorimetric readout distinct from at least one of the colorimetric signals (e.g., a yellow colorimetric signal and a blue colorimetric signal combine to produce a green colorimetric readout). The function may be described by Boolean logic. For example, one or more signals and/or one or more absences of signal may be analogous to Boolean “true” or “false” values. These values may be processed using Boolean logic, analogous to a logic gate (e.g., NOT, AND, NAND, OR, XOR, etc.).

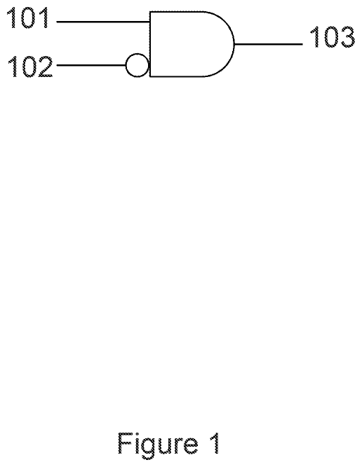

The Boolean logic gate may require that at least one signal is a “true” value, that at least one signal is a “false” value, a “true” value and a “false” value, all signals being “true” values, all signals being “false” values, or some combination of signals being “true” and “false” values. For example, FIG. 1 depicts an exemplary logic gate where the lack of sensing of one or more sensors (a logical negation) is needed to create a Boolean true output. In the depicted logic gate, a first signal 101 is provided by a first sensor and no signal (e.g., the absence of a signal) is provided from a second sensor 102. In this example the first signal 101 in the absence of a second signal 102 leads to a sensor output 103. That is, a Boolean logic gate may require one “true” value and one “false” value to provide a “true” output signal. Such a logic gate may be coupled with a first sensor responsive to a particular analyte A and a second sensor responsive to particular analyte B. The first sensor may provide a “true” signal in the presence of A, and the second sensor may independently provide a “true” signal in the presence of B. Thus, if the first sensor detects A but the second sensor does not detect B or if the second sensor detects B but the first sensor does not detect A, the logic gate will provide a “true” output signal. However, if the first sensor detects A and the second sensor detects B, then the logic gate will not provide a “true” output signal in this example.

In some embodiments, a plurality of logic gates or colorimetric signal combinations may be used to process a plurality of signals from a plurality of sensors. In certain embodiments, processing by the logic gates or combination of colorimetric signals does not occur separately from one another. A first logic gate of the plurality of logic gates may process some or all of the plurality of signals simultaneously, nearly simultaneously, or in conjunction with a second logic gate of the plurality of the logic gates. In certain embodiments, a first colorimetric signal is not separated from a second colorimetric signal or is provided in fluid communication to a second colorimetric signal to provide a colorimetric readout. For example, a yellow colorimetric signal in response to a first analyte may be combined directly with a blue colorimetric signal in response to a second analyte to provide a green colorimetric readout. In some embodiments the first colorimetric signal is not separated from a second colorimetric signal or is provided in fluid communication to a second colorimetric signal that is a negative signal to provide a colorimetric readout. For example, a yellow colorimetric signal in response to a first analyte may be combined directly with a clear colorimetric signal in response to the absence of a second analyte to provide a yellow colorimetric readout. In some embodiments, the first and second colorimetric signal directly combined may both be negative. For example, a red colorimetric signal in response to the absence of a first analyte may be combined directly with a clear colorimetric signal in response to the absence of a second analyte to provide a red colorimetric readout. In certain embodiments, the logic gates are in close physical proximity with one another. For example, in certain embodiments, the logic gates are not partitioned into separate channels or regions of a microfluidic device. Some or all of the logic gates may be contained in a homogeneous assay. In certain embodiments the logic gates do not require a predetermined order to process the plurality of signals. The logic gates may produce a consolidated signal. In certain embodiments the consolidated signal is produced by processing all, most, many, or some possible combinations of the logic gates. In preferred embodiments, the logic gates produce a consolidated signal (e.g. a colorimetric output) by processing most of the possible combinations of the logic gates. In preferred embodiments, processing by the logic gates requires does not require providing additional analytes or reagents through channels to create the final output.

In certain embodiments the calibration of one or more sensor may act as a threshold to qualify a signal provided by the sensor to be a Boolean “true” value. In certain embodiments, the threshold or thresholds of one or more sensor coupled with a Boolean logic gate will prevent outputs of “true” values where one or more of a plurality of input signals are weak.

The processing may provide a processed signal, preferably a colorimetric readout, as an output that may be displayed on the display. The processed signal may provide a qualitative indication of the accumulated exposure to one or more analytes. The processed signal may provide a qualitative indication of the real-time or near-real-time exposure to one or more analytes. The processed signal may provide a qualitative indication of a condition (e.g., a biological condition) of interest of a subject.

The technology may provide one or more readily understood signals. The signal(s) may indicate a qualitative measurement or a quantitative measurement. The signal(s) may comprise one or more processed signals. In preferred embodiments the readily understood signal is a single colorimetric readout. The one or more processed signals may be detectable by an unaided human eye. The one or more processed signals may be a colorimetric readout. In certain embodiments, the readout can be incorporated into a display (e.g. energy bars) where the output of each atomic component of the display (e.g. a single energy bar) can have an positive or negative colorimetric state (e.g. pink or blue) as the result of the compounding of all the colors emitted by the sensors composing the atomic component.

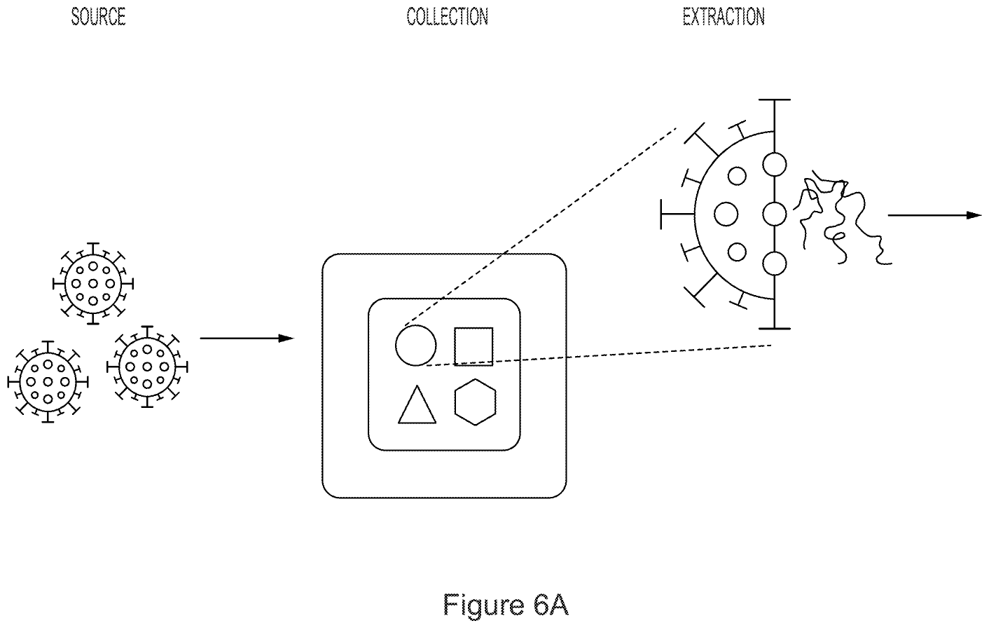

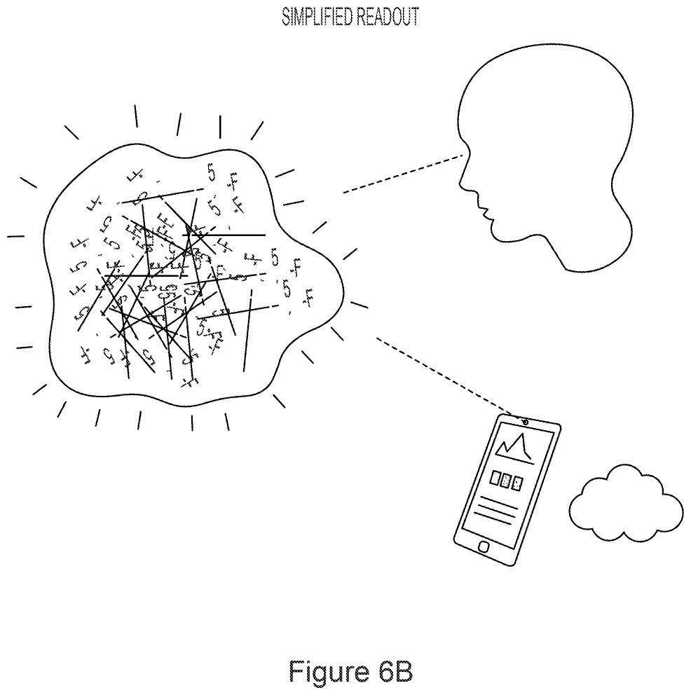

A reference, such as a reference color, may be used to aid a user in identifying or interpreting the one or more readily understood signals. The colorimetric readout may be the result of a direct interaction. For example, aggregation of gold nanoparticles in the presence of salt may result in a change color from red to blue. The colorimetric readout may be indirectly caused by an interaction. For instance, where a sensing modality of a sensor interacts with an analyte of interest, and the resulting signal is processed to produce an output signal, the output signal or a subsequent signal may result in the colorimetric readout. In certain embodiments, the application of UV light may be used to produce or detect the one or more signals. As depicted in FIG. 6B, the readout may be a simplified report or a directly observable signal.

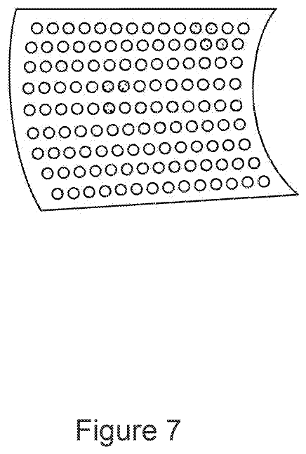

The one or more analyte of interest may be any molecule, particle, unit, or cell. The one or more analytes of interest may be obtained from any source. For example, the one or more analytes of interest may be obtained from human skin (e.g., dead skin cells, the skin microbiome, etc.), fecal matter, and swabs of oral cavities. Collection of the one or more analytes of interest may be performed using an object that also provides another function. For example, the one or more analytes of interest may be obtained using tissue papers (e.g., facial tissue paper, toilet paper, kitchen papers, paper towels, paper napkins, etc.), undergarments (e.g., underwear), diapers, and other clothing. In certain embodiments, the object used to collect the one or more analytes of interest may comprise the indicator of the instant technology or a portion of the indicator of the instant technology. FIG. 7 shows an exemplary embodiment of a tissue paper comprising the indicator of the instant technology that can be used to collect the one or more analytes of interest. Upon the presence of a signal, individual regions of the tissue paper may change color as depicted in FIG. 8. Further, each sensor adhered to the tissue paper may be removable.

In certain embodiments, the analyte of interest is a single-stranded nucleic acid (e.g., DNA, RNA, etc.). In certain embodiments, the analyte of interest may be obtained from the skin of a person (e.g., a person wearing an indicator). In embodiments where the analyte of interest is from the skin of a person, the analyte of interest may be from the microbiome of the person and/or free analytes (e.g., nucleic acids or proteins existing outside of a cell or viral membrane).

FIG. 6A depicts an exemplary embodiment wherein one or more analyte of interests is extracted from a biological source. In certain embodiments, the analyte of interest is obtained by extracting the contents of a cell. For example, the analyte of interest may be an analyte contained in an organismal cell (e.g., a DNA fragment, a protein, other biomolecules, etc.) which is extracted through ex-situ cell membrane lysing. The lysing process may be used to break down the external cell membrane as well as other cellular compartments, thereby releasing the one or more analytes of interest for detection by an indicator. It is well known in the art, what lysing agents may be used, including detergents and various salt combinations in order to extract analytes of interest.

In certain embodiments, the analyte of interest is obtained from saliva or another bodily fluid. The saliva or other bodily fluid may be obtained directly from a subject or from droplets or micro-droplets in the air on a surface (e.g., a metal, a glass, a plastic). In certain embodiments, the analyte of interest present in a saliva or bodily fluid may be stabilized either by chemical means or by mechanical means (e.g., centrifuge-free use of silicate nanoparticles).

In certain embodiments, detection of or indication of the absence of one or more analytes of interest may be used to evaluate various health related conditions such as cancers, inflammation, skin disorders, age related changes, the presence of a virus (e.g. covid-19), etc.

In certain embodiments, the detection of or indication of the absence of one or more analytes of interest (e.g., nucleic acids, proteins, etc.) from the microbiome (e.g., the human microbiome) may be used to the predict cancers, inflammation disorders, or other health conditions including age related changes, or the presence of a virus (e.g. covid-19). For example, human bodies are continuously exposed to microbial cells and the microbial cell byproducts are known to include toxic metabolites. Circulation of toxic metabolites may contribute to cancer onset. In addition, microbes associated with tumor development may migrate throughout the human body. Several metagenomics studies showed that dysbiosis in the commensal microbiota is associated with inflammatory disorders and various cancers. For instance, the most recognizable link has been found between the microbiome and cancer via the immune system. Wen-Ming Wang, Hong-Zhong Jin, Skin microbiome: An actor in the pathogenesis of psoriasis, Chin Med J (Engl). 2018: 131(1), 95-98.

As an example, gut microflora have been found to be associated with gastrointestinal cancer. The most prominent is association of Helicobacter pylori with gastric adenocarcinoma and gastric mucosaassociated lymphoid tissue lymphoma. The bacterium Campylobacter jejuni and Salmonella typhi have also been associated with small intestine lymphoma and gall bladder cancer, respectively. In these cases, chronic inflammation at the tumor site induces carcinogenesis. Other reports support that gut microflora play protective roles as well against cancer. Helicobacter pylori has been shown to reduce the risk of esophageal squamous cell carcinoma, and pancreatic cancer. This suggests that a balanced and precise monitoring of microbiome is necessary for a robust immune response. Detection or the indication of the absence of any of these exemplary microflora may be used to predict or determine the health condition of a subject.

The detection or indication of the absence of microbes in the human microbiome or its composite molecules (e.g., nucleic acids and protein) can be used to predict various skin conditions such as hydration, serum level, and sebum level. To this end, certain embodiments of the sensor could be designed to respond to human microbiome specific nucleic acids. As an example, it has been shown that the proportion of the phylum Actinobacteria and the genus Propionibacterium significantly decreased with increasing skin hydration levels on the forehead. Souvik Mukherjee, Rupak Mitra, Arindam Maitra, Satyaranjan Gupta, Srikala Kumaran, Amit Chakrabortty, Partha P. Majumder, Sebum, serum, and hydration levels in specific regions of human face significantly predict the nature and diversity of facial skin microbiome, Sci Rep. 2016: 6, 36062. This study measured sebum and hydration from forehead and cheek regions of healthy female volunteers (n=30) and metagenomic DNA from skin were sequenced. In this case, 34 phyla were identified (mostly Actinobacteria, Firmicutes, Proteobacteria and Bacteroidetes) and 1000 genera were identified (mostly Propionibacterium, Staphylococcus, Streptococcus, Corynebacterium and Paracoccus). Analysis showed that cheek sebum level was the most significant predictor of microbiome composition and diversity followed by forehead hydration level. These studies showed that the nature and diversity of facial skin microbiome should be determined by site-specific lipid and water levels which highlight the importance of using such a device for these cases. Detection or the indication of the absence of any of these exemplary microbes may be used to predict or determine the health condition of a subject.

The detection or indication of the absence of microbes in the human microbiome or its composite molecules (e.g., nucleic acids and protein) can be used to evaluate skin inflammation. Elizabeth A Grice and Julia A Segre, The skin microbiome, Nature Reviews Microbiology 2011: 9, 244-253. It has been shown that the skin microbiome greatly impacts the human immune functions. The mechanisms probably include inhibiting the growth of pathogenic microbes, enhancing host innate immunity, and educating adaptive immunity. In a study, it has been shown that S. epidermidis can inhibit S. aureus biofilm formation. In another study, after inoculation of the upper arm, swabs were taken at multiple time points for Haemophilus ducreyi. Papules either spontaneously resolved or progressed to pustules, with the microbiomes differing between the two groups. Proteobacteria, Bacteroidetes, Micrococcus, Corynebacterium, Paracoccus, and Staphylococcus species were more abundant at pustule-forming sites, whereas resolved sites had a greater abundance of Actinobacteria and Propionibacterium species. These data illustrate a crucial role for commensal bacteria in the host immune defense against pathogens and the importance of using wearable sensor for monitoring them. Detection or the indication of the absence of any of these exemplary microbes may be used to predict or determine the health condition of a subject.

The detection or indication of the absence of microbes in the human microbiome or its composite molecules (e.g., nucleic acids and protein) can be used to evaluate different skin disorders such as acne, psoriasis, and eczema. Recent studies showed that the presence of human skin microbiome and their composition is directly related to many disorders such as atopic dermatitis, psoriasis, and acne vulgaris. Elizabeth A Grice, The skin microbiome: potential for novel diagnostic and therapeutic approaches to cutaneous disease, Semin Cutan Med Surg. 2014: 33(2), 98-103. Detection or the indication of the absence of any of these exemplary microbes may be used to predict or determine the health condition of a subject.

The detection or indication of the absence of microbes in the human microbiome or its composite molecules (e.g., nucleic acids and protein) can be used to evaluate the presence of a genetic predisposition in the wearer. As an example, the influence of microbiomes on various dermatologic diseases has been investigated by sequencing the 16S rRNA-gene to analyze the correlation of skin bacterial microbiome in several skin disease states, including psoriasis and skin ulcers. These studies revealed that toxigenic strains were significantly increased in patients with skin disorders compared to healthy controls. Heidi H. Kong, Skin microbiome: genomics-based insights into the diversity and role of skin microbes, Trends Mol Med. 2011, 17(6), 320-328. Detection or the indication of the absence of any of these exemplary microbes may be used to predict or determine the health condition of a subject.

The detection or indication of the absence of microbes in the human microbiome or its composite molecules (e.g., nucleic acids and protein) can be used as an age indicator to quantify age related changes. Such use may be based on studies where an alteration is observed in the skin microbiome with aging by analyzing bacterial 16S rRNA gene sequencing. The analyses revealed that the alpha species was significantly higher in the older than the younger group, while the beta diversity in the overall structure significantly differed particularly for the forearm and scalp microbiomes between the two age groups. In addition, taxonomic profiling showed a significant reduction in the relative abundance of the majority skin genus Propionibacterium in the cheek, forearm, and forehead microbiomes of the older adults. Nakako Shibagaki, Wataru Suda, Cecile Clavaud, Philippe Bastien, Lena Takayasu, Erica Iioka, Rina Kurokawa, Naoko Yamashita, Yasue Hattori, Chie Shindo, Lionel Breton, Masahira Hattori, Aging-related changes in the diversity of women's skin microbiomes associated with oral bacteria, Sci Rep. 2017: 7, 10567.

### EXAMPLES

### Example 1

### Detection of RNA Targets by Molecular Beacons

RNA targets may be detected using the instant technology employing one or more molecular beacons.

The target may be an RNA target. Partially complementary nucleic acids are added. The first partially complementary nucleic acid (F1₁) and the second partially complementary nucleic acid (F1₂), partially hybridize to the RNA target. This forms a probe-target complex.

A pool of molecular beacons is added to the probe-target complex. The molecular beacons each have a spacer (S) and a catalytic loop (C). The molecular beacon has a fluorophore (F) attached at one terminal end and a quenching molecule (Q) at the opposite end that are in proximity when the beacon is free.

The molecular beacon is partially complementary to the sequence of the first partially complementary nucleic acid and the second partially complementary nucleic acid that are not complementary to the RNA target. Thus, the molecular beacon can co-hybridize with the bound probes based on its own partial complementarity. This forms a four-nucleic acid complex.

Upon hybridization between the molecular beacon and the probe-target complex, the four-nucleic acid complex reveals a cite for enzymatic restriction (X) on the molecular beacon. At this enzymatic restriction cite, enzymatic cleavage will occur. This leads to separating the molecular beacon end attached to the fluorophore and the end attached to the quencher.

Upon release from the partially complementary nucleic acids, the fluorophore end of the molecular beacon and the quencher end of the molecular beacon will diffuse, thereby separating the fluorophore from the quencher and resulting in a detectable signal.

Because multiple molecular beacons can be processed by a single RNA target, the signal may be amplified in proportion to the number of RNA targets present in a sample.

### Example 2

### Detection of SARS-CoV-2 Virus

The instant technology may be used to detect the presence of the SARS-CoV-2 virus.

A target nucleic acid from the genomic RNA of the SARS-CoV-2 virus is provided to the indicator. The indicator possesses a sensor comprising reporter strand 1 and reporter strand 2 in nearly molecular equivalents.

Report strand 1 possesses a fluorophore (FAM). Reporter strand 2 possesses a quencher (BHQ1).

Report strand 1 and reporter strand 2 have target anchors. The target anchors are partially complementary to the target nucleic acid. The partially complementary target anchors hybridize with the target sequence.

Reporter strand 1 and report strand 2 both have a first domain (domain 1) and a second domain (domain 2). The first domain of reporter strand 1 is complementary to the first domain of reporter strand 2. These first domains hybridize to one another. The second domain of reporter strand 1 is complementary to the second domain of reporter strand 2. These second domains hybridize to one another. The reporter strands are able to hybridize at their first domains and further at their second domains.

Reporter strand 1 and reporter strand 2 both have a non-complementary sequence. These non-complementary sequences separate the first domain and the second domain of the same reporter strand. These non-complementary sequences do not hybridize to one another.

In the absence of the SARS-CoV-2 virus gRNA target sequence, the two reporter strands will hybridize with one another at the first domains and the second domains. This hybridization will bring the quencher into proximity with the fluorophore. However, the non-complementary regions will not produce a catalytic loop for cleavage. The resulting quenching the emissions of the fluorophore is a detectable signal indicating the absence of the SARS-CoV-2 virus.

In the presence of the SARS-CoV-2 virus gRNA target sequence, the reporter strands will further partially hybridize with the target sequence by the target anchors. This additional hybridization will further stabilize the reporter strand 1-reporter strand 2 hybridization. This will allow for catalytic loop formation at the non-complementary region of reporter strand 2.

In the presence of DNAzyme and about 1 mM Zn²⁺, restriction occurs at the catalytic loop at the non-complementary region of reporter strand 2. The resulting fragments of reporter strand 2 along with the BHQ1 quencher dissociates from the hybrid complex, thereby increasing the detectable fluorescent emission from the fluorophore, FAM. The increase detectable increase of emissions of the fluorophore acts as an indicator of the presence of the SARS-CoV-2 virus.

### Example 3

Using the indicator of the technology can be employed to combine sensors to identify the same or very similar conditions across subjects despite having a high inter-subject variability in the marker compositions found from the skin. For example, skin hydration correlates with high levels of at least one of six phyla present on the human forehead. Mukherjee, S., Mitra, R., Maitra, A, Gupta, S., Kumaran, S., Chakrabortty, A, & Majumder, P. P. (2016). Sebum and Hydration Levels in Specific Regions of Human Face Significantly Predict the Nature and Diversity of Facial Skin Microbiome. Scientific Reports, 6, 36062. Retrieved from http://dx.doi.org/10.1038/srep36062

Here, the presence of a marker creates a compounded colorimetric readout by combining a plurality of sensors into the same device. The sensors are not separated from each other. Each sensor is sensing for a sequence of interest (SOI) that uniquely identifies each of the six phyla where each SOI is common to all or mostly all organisms within that phylum, while not present in all or mostly all of the organisms belonging to other phyla. The colorimetric output of each sensor is calibrated to be additive, acting as a logical OR gate. Color calibration of each sensor considers thresholds that qualify the input as a Boolean ‘true’ while preventing scenarios such as all inputs showing a weak signal—Boolean ‘false’ inputs—but outputting a Boolean ‘true’.

Other examples include situations where the compounded colorimetric output may be subtractive. For example, the presence of species A correlates positively with a certain condition but the co-presence of species B correlates negatively with that particular health condition, thus a strong colorimetric output will be visible to the user only when species A is relatively abundant without species B being also abundant. An exemplary usage of a logic gate is seen in FIG. 1.

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein.

All references and publications recited are incorporated by reference, including:
[1] Wen-Ming Wang, Hong-Zhong Jin, Skin microbiome: An actor in the pathogenesis of psoriasis, Chin Med J (Engl). 2018: 131(1), 95-98.
[2] Souvik Mukherjee, Rupak Mitra, Arindam Maitra, Satyaranjan Gupta, Srikala Kumaran, Amit Chakrabortty, Partha P. Majumder, Sebum and hydration levels in specific regions of human face significantly predict the nature and diversity of facial skin microbiome, Sci Rep. 2016: 6, 36062.
[3] Elizabeth A. Grice and Julia A. Segre, The skin microbiome, Nature Reviews Microbiology 2011: 9, 244-253.
[4] Elizabeth A. Grice, The skin microbiome: potential for novel diagnostic and therapeutic approaches to cutaneous disease, Semin Cutan Med Surg. 2014: 33(2), 98-103.
[5] Heidi H. Kong, Skin microbiome: genomics-based insights into the diversity and role of skin microbes, Trends Mol Med. 2011, 17(6), 320-328.
[6] Nakako Shibagaki, Wataru Suda, Cecile Clavaud, Philippe Bastien, Lena Takayasu, Erica Iioka, Rina Kurokawa, Naoko Yamashita, Yasue Hattori, Chie Shindo, Lionel Breton, Masahira Hattori, Aging-related changes in the diversity of women's skin microbiomes associated with oral bacteria, Sci Rep. 2017: 7, 10567.
[7] BS Alladin-Mustan, C J Mitran, J M Gibbs-Davis, Achieving room temperature DNA amplification by dialing in destabilization. Chem Commun (Camb). 2015 Jun. 4; 51(44):9101-4. doi: 10.1039/c5cc01548k. PMID: 25920515.

1. An indicator comprising:
a substrate; and
an interface disposed on the substrate, the interface comprising:
a first sensor responsive to a first analyte, the first sensor comprising a first sensing modality, the first sensing modality being a biochemical modality or a chemical modality, and the first sensor being configured to provide a first colorimetric signal upon interaction with the first analyte;
a second sensor responsive to a second analyte, the second sensor comprising a second sensing modality, the second sensing modality being a biochemical modality or a chemical modality, and the second sensor being configured to provide a second colorimetric signal upon interaction with the second analyte; and
a display configured to display a colorimetric readout.

2. The indicator of claim 1, wherein the interface is configured to combine the first colorimetric signal and the second colorimetric signal to output the colorimetric readout.

3. The indicator of claim 2, wherein the interface is configured to compound the first colorimetric signal.

4. The indicator of claim 2, wherein the interface is configured to dilute the first colorimetric signal.

5. The indicator of claim 2, wherein the colorimetric readout is substantially identical to the first colorimetric signal.

6. The indicator of claim 2, wherein the colorimetric readout is distinct from the first colorimetric signal, and wherein the colorimetric readout is distinct from the second colorimetric signal.

7. The indicator of claim 1, wherein the colorimetric readout is configured to have an intensity, wherein the intensity is proportional to one or more of the concentration of the first analyte, the concentration of the second analyte, the amount of the first analyte, and the amount of the second analyte.

8. The indicator of claim 1, wherein the substrate is a polymeric substrate.

9. The indicator of claim 1, wherein the colorimetric readout is configured as a single colorimetric readout.

10. The indicator of claim 1, wherein the first sensing modality is a cell-free modality, a whole-cell modality, or a nanoparticle modality.

11. The indicator of claim 10, wherein the first analyte is single-stranded DNA.

12. The indicator of claim 1, wherein the display comprises a logical gate, the logic gate is configured to output the colorimetric readout, wherein the logic gate is responsive to a predetermined logical condition.

13. The indicator of claim 12, wherein the predetermined logical condition is at least one of the first colorimetric signal and the second colorimetric signal being a Boolean true signal.

14. The indicator of claim 12, wherein the predetermined logical condition is at least one of the first colorimetric signal and the second colorimetric signal being a Boolean false signal.

15. The indicator of claim 12, wherein the predetermined logical condition is the first colorimetric signal being a Boolean true signal and the second colorimetric signal being a Boolean false signal.

16. The indicator of claim 12, wherein the indicator further comprises a second logic gate, wherein the second logic gate is responsive to a second predetermined logical condition, and wherein the first logic gate and the second logic gate are configured to output the colorimetric readout.

17. The indicator of claim 12, wherein the first analyte is an antigen, wherein the first sensing modality comprises one or more antibodies configured to bind the antigen.

18. The indicator of claim 12, wherein the first analyte is an antibody, wherein the first sensing modality comprises one or more antigens configured to bind the antigen.

19. The indicator of claim 12, wherein the first analyte is a nucleic acid, wherein the first sensing modality comprises one or more nucleic acids,
wherein the one or more nucleic acids of the first sensing modality is configured to interact with the first analyte, and
wherein the one or more nucleic acids of the first sensing modality is configured to interact with the first analyte based on one or more of intercalating agents, enzymes, beacons, or salts.

20. The indicator of claim 19, wherein the one or more nucleic acids of the sensing modality is configured to interact with the first analyte based on enzymes, and wherein at least one of the one or more nucleic acids of the first sensing modality is configured to have a G-hairpin conformation.

21. (canceled)

22. The indicator of claim 17, wherein the at least one of the first analyte and the second analyte is a nucleic acid.

23. The indicator of claim 17, wherein the first sensing modality comprises one or more bioreceptors.

24. The indicator of claim 23, wherein the first sensing modality comprises one or more nucleic acids.

25. (canceled)

26. The indicator of claim 23, wherein the first sensing modality further comprises nanomaterials, wherein the nanomaterials constitute a host matrix, and wherein the one or more bioreceptors are disposed on the host matrix.

27. The indicator of claim 26, wherein the nanomaterials are carbon nanomaterials.

28. The indicator of claim 1, wherein the first sensor is in fluid communication with the second sensor.

29. The indicator of claim 1, wherein the first sensor is not separated from the second sensor.

30. The indicator of claim 1, wherein the interface is configured to be wearable on the skin of a subject or on a surface.

31. (canceled)

32. The indicator of claim 1, further comprising an adhesive layer.

33. The indicator of claim 1, further comprising a membrane layer.

34. (canceled)

35. The indicator of claim 33, wherein the membrane layer is porous.

36. (canceled)

37. The indicator of claim 1, wherein the first analyte is derived from a microbiome of a subject.

38.-39. (canceled)

40. The indicator of claim 1, wherein the first analyte and the second analyte are different analytes.

41. The indicator of claim 1, wherein the first analyte and the second analyte are the same analyte.

42. A method for determining exposure to at least one analyte, the method comprising:
providing an indicator comprising:
a substrate;
an interface disposed on the substrate, the interface comprising:
a first sensor responsive to a first analyte, the first sensor comprising a first sensing modality, the first sensing modality being a biochemical modality or a chemical modality, and the first sensor being configured to provide a first colorimetric signal upon interaction with the first analyte;
a second sensor responsive to a second analyte, the second sensor comprising a second sensing modality, the second sensing modality being a biochemical modality or a chemical modality, and the second sensor being configured to provide a second colorimetric signal upon interaction with the second analyte; and
a display configured to display a colorimetric readout;
determining exposure to at least one of the first analyte and the second analyte; and
displaying the colorimetric readout on the display.

43.-87. (canceled)

88. A system for determining exposure to at least one analyte, the system comprising:
an indicator comprising:
a substrate; and
an interface disposed on the substrate, the interface comprising:
a first sensor responsive to a first analyte, the first sensor comprising a first sensing modality, the first sensing modality being a biochemical modality or a chemical modality, and the first sensor being configured to provide a first signal upon interaction with the first analyte;
a second sensor responsive to a second analyte, the second sensor comprising a second sensing modality, the second sensing modality being a biochemical modality or a chemical modality, the second sensor being configured to provide a second signal upon interaction with the second analyte; and
a reactive solution;
wherein the reactive solution is configured to interact with one or more of the first signal and the second signal to produce a colorimetric readout.

89.-124. (canceled)

125. The indicator of claim 1, wherein the display is configured to be reversible, and wherein the colorimetric readout from the display is configured to be eliminable.
